(19) United States
(12) Patent Application Publication
(10) Pub. No.: US 2011/0061658 A1
(43) Pub. Date: Mar. 17, 2011

(54) OROPHARYNGEAL DEVICES FOR USE IN VENTILATING PATIENTS

(76) Inventors: Robert Koorn, Redding, CT (US); Eric Kitain, Armonk, NY (US); Vladimir V. Koltchine, Redding, CT (US)

(21) Appl. No.: 12/719,615

(22) Filed: Mar. 8, 2010

Related U.S. Application Data
(60) Provisional application No. 61/233,193, filed on Aug. 12, 2009.

(51) Int. Cl. A61M 16/00; B29C 47/02

(52) U.S. Cl. 128/207.14; 264/271.1

(57) ABSTRACT

An oropharyngeal device, for example, an endotracheal tube or a laryngeal mask airway, intended for insertion into a patient includes a tube portion defining a lumen which permits flow of a ventilating gas to the patient who may be under anesthesia for performance of surgery. The device includes a reinforcement member in a bite-resistant zone, which keeps the lumen open during patient biting to prevent occlusion of the lumen and/or interruption of the flow of ventilating gas. The reinforcement member can have a number of adjacent or closely-spaced wire turns or another suitable structure, and can cooperate with the tube portion to provide good bite-resistance. The tube portion optionally can be yieldable in the bite-resistant zone and provide cushioning during biting to inhibit dental or other damage.

### CROSS-REFERENCE TO A RELATED APPLICATION

This application claims the benefit of provisional patent application No. 61/233,193, filed on Aug. 12, 2009, the disclosure of which is incorporated by reference herein.

### STATEMENT REGARDING FEDERALLY SPONSORED RESEARCH OR DEVELOPMENT

(Not Applicable.)

The present invention relates to oropharyngeal devices useful in ventilating patients, for example, endotracheal tubes, laryngeal mask airways, and other comparable devices. The invention also relates to methods for manufacturing and using orolaryngeal devices.

### BACKGROUND OF THE INVENTION

An orolaryngeal device is a hollow or tubular device which can be inserted into the mouth and pharynx of a subject, who may be a patient receiving medical care, to provide a flow of air or other gas to a patient. Because such orolaryngeal devices generally include a tube portion passing between the patient's teeth, they are vulnerable to damage or obstruction caused by the patient biting down on the device. Endotracheal tubes and laryngeal mask airways are examples of orolaryngeal devices, each of which is further described herein. Other examples will be known or apparent to a person of ordinary skill in the art, in light of this disclosure, or will become known or apparent in the future, as the art develops.

One known embodiment of an endotracheal tube comprises a hollow, cylindrically shaped tube that can be inserted through the mouth, through the vocal cords of the larynx, and into the trachea of a patient. Once placed in position, the endotracheal tube can be used to administer oxygen, air or other gases to the patient either by the patient's own spontaneous breathing or by using an artificial ventilation device. An endotracheal tube can also help prevent entry into the lungs of stomach contents or other foreign material. Endotracheal tubes are often used to facilitate surgery and may be inserted and manipulated by highly-trained and experienced anesthesiologists. Sometimes, for example, in emergency situations, an endotracheal tube may be used by a less-skilled technician or other operator. Because the endotracheal tube passes between the patient's teeth, usually but not necessarily the incisors, endotracheal tubes can be vulnerable to obstruction resulting from the patient biting down on the endotracheal tube.

A laryngeal mask airway (sometimes referred to as a “LMA stem”) is usually shorter than an endotracheal tube and comprises a less invasive airway management tool which is not placed into the trachea of the patient. Rather, a laryngeal mask airway can be placed within the mouth and pharynx to define an unobstructed breathing or ventilation airway past the patient's tongue and other soft tissues into the patient's upper airway. Typically, a laryngeal mask airway can comprise a large bore stem that traverses the incisors as it exits the mouth. Here the laryngeal mask airway is also prone to obstruction arising from the patient biting down on the stem of the airway. Furthermore, the size of the laryngeal mask airway may cause it to be difficult to remove under bite-down conditions.

The art contains various proposals for endotracheal tubes. For example, U.S. Pat. No. 5,722,395 to Kolobow describes an ultra thin walled wire reinforced endotracheal tubing that includes a thin walled tubing comprising a polymeric material having a spring material incorporated therewith. As described, utilization of the spring wire material in combination with polymeric material results in a reduced wall thickness which results in a significant decrease in resistance to air flow through the endotracheal tubing.

The art also describes drawbacks which may be associated with some endotracheal tubes having spring wire reinforcement, which tubes are also known in the art as “armored tubes” and “flexometallic tubes.” For example, Malhotra et al., in the Indian Journal of Anesthesia (2007); 51(5): 432-433, describe a case of a 40-year-old male undergoing surgery in the prone position under general anesthesia with a spiral-embedded endotracheal tube. As described in the publication, a patient bite-down on the tube created a permanent deformity resulting in occlusion, leading to hypoxia and desaturation. To avoid such problems, a bite block can be employed to protect an endotracheal tube from closure caused by patient bite-down.

For example, U.S. Patent Application Publication No. 2002/0092526 by Bertoch et al. describes a securing device for an endotracheal tube that preferably includes a shield having an opening through which the endotracheal tube can pass and a clamp mounted on the shield for holding the endotracheal tube. As described, a bite block for preventing occlusion of the endotracheal tube by a patient's teeth may be mounted on an opposite surface of the shield from the clamp.

Also, U.S. Patent Application No. 2009/0133701 by Brain describes a laryngeal mask airway device for insertion into a patient to provide an airway passage to the patient's glottic opening, which can employ a bite block (reference numeral 44 in FIG. 12).

The foregoing description of background art may include insights, discoveries, understandings or disclosures, or associations together of disclosures, that were not known to the relevant art prior to the present application but which were provided by the present application and its teachings. Some such contributions of the present invention may have been specifically pointed out herein, whereas other such contributions of the invention will be apparent from their context.

### BRIEF SUMMARY OF THE INVENTION

The present invention provides, inter alia, an oropharyngeal device useful in supplying a ventilating gas to a patient's airway, which device can resist bite-down by the patient and can maintain patency of the airway in various situations.

The present invention provides, in one aspect, an oropharyngeal device which is useful in ventilating a patient by supplying a ventilating gas to an airway of the patient. The oropharyngeal device generally comprises a proximal end connectable with a source of ventilating gas and a distal end disposable within the airway of the patient while the proximal end is located externally of the patient. The oropharyngeal device provided in this aspect of the invention also generally comprises a tube portion extending between the proximal end and the distal end of the device, which tube portion defines a lumen permitting ventilating gas to flow through the oropharyngeal device from the proximal end to the distal end.

The tube portion can comprise a tube wall formed of a polymeric material, for example, a flexible polymeric or flexible resilient polymeric material, or other suitable material, and has a bite-resistant zone located intermediately of the tube portion between the proximal end and the distal end of the tube portion. The bite-resistant zone can comprise a zone of the tube portion which is subject to engagement between the teeth of the patient during use.

The orolaryngeal device also comprises a reinforcement member extending around the lumen in the bite-resistant zone and is configured to keep the lumen open during patient biting to permit the ventilating gas to flow through the lumen. In some embodiments, the reinforcement member defines the bite-resistant zone. Optionally, the tube wall can provide cushioning, for example, the tube wall can be yieldable in response to biting pressure.

Although various structure and materials can be used, the reinforcement member can comprise a resilient wire reinforcement extending in multiple turns around the lumen in the bite-resistant zone. The wire reinforcement can have various structures or configurations, some of which are described herein. For example, turns of a wire reinforcement can be spaced closely together to resist biting pressure and keep a lumen open during biting and thus permit a ventilating gas to flow through the lumen during patient biting. Other suitable structures of the wire reinforcement will be known or apparent to a person of ordinary skill in the art, in light of this disclosure, or will become known or apparent in the future, as the art develops.

For some embodiments of orolaryngeal devices, for example, endotracheal tubes or laryngeal mask airways, use of an orolaryngeal device as presently described herein can avoid the need for a bite block, or the like, which can simplify surgical procedures and can enhance safety.

In addition, an orolaryngeal device can comprise a distal support member for the tube wall and a proximal support member for the tube wall. The distal and proximal support members can be located distally and proximally adjacent to a reinforcement member respectively and can be configured to be capable of supporting the tube wall to permit flexing of the tube portion about an axis transverse to the tube portion and prevent kinking. Absent such support structure, kinking may occur in portions of some flexible tubular orolaryngeal devices when flexed. Accordingly, such support structure is referred to herein as “a kink-inhibiting structure,” of which widely-spaced wire turns are an example.

In a further aspect, the invention provides a manufacturing process for making an orolaryngeal device such as described herein. The process generally comprises preforming, preassembling or predisposing a reinforcement member in an appropriate manner; and building a tube portion outside, inside or around the preformed, preassembled or predisposed reinforcement member, by extrusion, by molding, by deposition, or in another suitable manner. One or more distal end components, proximal end components, and any other desired components of the orolaryngeal device then can be assembled to complete the device.

Other manufacturing processes can be employed, some of which are also described herein, and others of which will be known or apparent to a person of ordinary skill in the art, in light of this disclosure, or will become known or apparent in the future, as the art develops.

### BRIEF DESCRIPTION OF THE SEVERAL VIEWS OF THE DRAWING

Some embodiments of the invention, and ways of making and of using the same, are described in detail herein and by way of example, with reference to the accompanying drawings (which are not necessarily drawn to scale with regard to any internal or external structures shown) and in which like reference characters designate like elements throughout the several views, and in which:

FIG. 1 is a plan view of one embodiment of an oropharyngeal device according to the teachings of the present application, which oropharyngeal device comprises an endotracheal tube;

FIG. 2 is a sectional view of a portion of the endotracheal tube shown in FIG. 1;

FIG. 3 is a plan view of another embodiment of an oropharyngeal device according to the teachings of the present application, which oropharyngeal device also comprises an endotracheal tube;

FIG. 4 is a sectional view of a portion of the endotracheal tube shown in FIG. 3;

FIG. 5 is a plan view of a further embodiment of an oropharyngeal device according to the teachings of the present application, which oropharyngeal device comprises a laryngeal mask airway; and

FIG. 6 is a sectional view of a portion of the laryngeal mask airway shown in FIG. 5.

### DETAILED DESCRIPTION OF THE INVENTION

The present invention provides, an oropharyngeal device, which can be in the form of an endotracheal tube and/or an laryngeal mask airway, either of which can be useful during general anesthesia, or in other circumstances.

In some embodiments, an endotracheal tube or a laryngeal mask airway can be internally reinforced with a reinforcement member, for example, a wire made of a metal, a metal alloy, carbon fibers, combinations thereof, or another suitable material. The metal wire can be coiled and the wire coils can be spaced and wound in a particular manner to provide bite-resistance. For example, in a section of the endotracheal tube or laryngeal mask airway that is likely to be adjacent to the patient's incisor teeth during use, the wire coils can be wound tightly or closely. In certain embodiments, the wire coils can be adjacent each other, mimicking a solid metal pipe. The tightly-wound coils can resist or prevent occlusion of the endotracheal tube or laryngeal mask airway during bite-down. Proximally and/or distally of the reinforcement member, for example, of the closely-spaced wire coils, a kink-inhibiting structure (e.g., widely-spaced wire coils) can be present, permitting flexing of the endotracheal tube or laryngeal mask airway while inhibiting kinking, which could occlude or obstruct the endotracheal tube or laryngeal mask airway. Each of the kink-inhibiting structures, for example, widely-spaced wire coils, can be contiguous with or separate from the reinforcement member. The kink-inhibiting structure(s) also can be formed from the same structure as the reinforcement member, for example, as a one-piece component.

Variations and modifications of these exemplary structures are possible, some of which are described herein and others of which will be known or apparent to a person of ordinary skill in the art, in light of this disclosure, or will become known or apparent in the future, as the art develops.

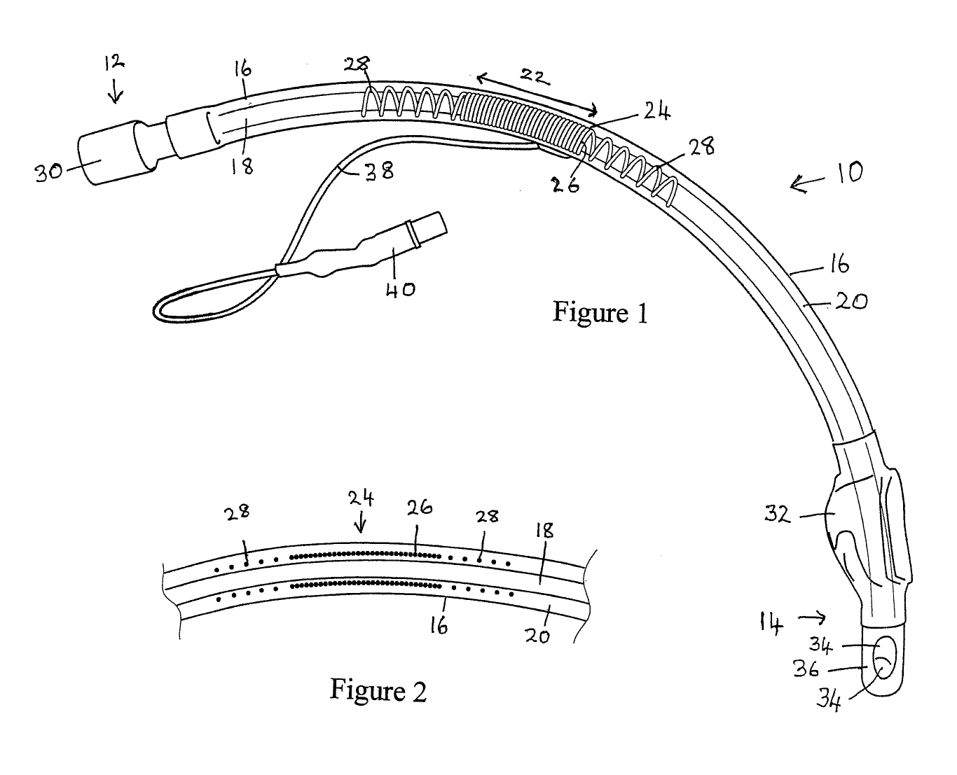

The exemplary endotracheal tube illustrated in FIGS. 1 and 2 comprises a hollow, cylindrically shaped tube that can be placed through the mouth, and through the vocal cords of the larynx, into the trachea of a patient. After placed in position, the endotracheal tube can be used to administer oxygen and other gases to the patient either by the patient's own spontaneous breathing or by using artificial ventilation devices.

Referring to the figures, the endotracheal tube 10 comprises two ends: a proximal end 12 and a distal end 14. The proximal end 12 can be connected with a source of ventilating gas (not shown) and can be disposed externally of a patient, or other subject. The distal end 14 can be disposed internally of the patient, for example, in or within an airway, such as the pharynx, trachea, or mouth of the patient. In such a disposition, the endotracheal tube can extend from the internally disposed distal end through the mouth and between the teeth of the patient to the externally disposed proximal end. Other possible useful dispositions of the endotracheal tube will be, or become, apparent to a medical practitioner or other user according to particular circumstances.

Again referring to FIGS. 1 and 2, the endotracheal tube 10 further comprises a tube portion 16 extending between the proximal end 12 and the distal end 14 of the endotracheal tube 10. The tube portion 16 defines a lumen 18 that is surrounded by a tube wall 20, which lumen 18 permits ventilating gas to flow through the endotracheal tube 10 from the proximal end 12 to the distal end 14.

A lumen can have any suitable shape and dimensions that will permit an adequate flow of oxygen, air or other gas from the proximal end to the distal end according to the intended use of the endotracheal tube. The lumen can have any suitable cross-sectional shape, for example, circular, oval, elliptical, rectangular, hexagonal, polygonal, or another shape. An angular cross-sectional shape, if employed, can be rounded, if desired, comprising one or more rounded sides or rounded angles or both. The cross-sectional shape and area of the lumen can be constant throughout the length of the tube portion. Alternatively, the cross-sectional shape and area of the lumen can vary along the length of the tube portion provided that delivery of ventilating gas, or of another service for which the endotracheal tube is intended, is not adversely affected.

Desirably, the lumen is unobstructed to promote delivery of oxygen, air or other gas to the patient. In some embodiments, the lumen can also permit the insertion of a suction tube or another useful airway deliverable device. In addition, the tube portion can have a smooth inner surface, for example, to provide low airflow resistance and facilitate laminar, non-turbulent gas flow through the lumen, if desired. The inner surface of the tube portion can be polished, coated or otherwise treated, or can be lined, for example, to reduce airflow resistance. In particular embodiments, the inner surface can be untreated.

In particular embodiments (not shown), a tube wall of a tube portion can have a composite structure. For example, an outer tubular layer can be disposed concentrically around an inner tubular layer and a reinforcement member can be present or embedded between the inner tubular layer and the outer tubular layer.

A tube wall can be formed partially, or entirely, of any suitable polymeric or other material, for example, a flexible polymeric material or a flexible resilient polymeric material. One suitable flexible resilient polymeric material is or comprises a medical grade polyvinyl chloride (PVC) polymer. Another suitable material is or comprises a silicone rubber. The tube wall material can be transparent or translucent, which can render an embedded reinforcement member and kink-inhibiting structure, if present, visible through the tube wall. Other suitable medical grade or other polymeric materials can be employed as would be recognized by a skilled artisan.

A tube wall of an endotracheal tube can have any suitable thickness according to the material employed and the intended application. For example, the tube wall can have a thickness greater than 0.5 mm, such as a thickness in the range of from about 0.5 mm to about 5 mm, or in the range of from about 1 mm to about 2 mm. As stated herein, a reinforcement member can be embedded in the tube wall, without increasing the thickness of tube wall. In some cases it may be desirable for an endotracheal tube to have a thin tube wall to optimize the airflow capacity of the lumen for a given outer diameter or other transverse dimension of the tube portion, and to be accommodated by the patient's anatomy.

A tube portion can have any suitable dimensions. The dimensions of the tube portion typically are selected according to the anatomy of the subject with whom the endotracheal tube is intended to be used. For example, the tube portion can have a length in the range of from about 150 mm to about 500 mm, or a length in the range of from about 200 mm to about 400 mm, or a length in the range of from about 250 mm to about 350 mm.

A tube portion can have a diameter or a major transverse dimension in the range of from about 3 mm to about 25 mm, or in the range of from about 5 mm to about 12 mm, or in the range of from about 6 mm to about 10 mm. In certain embodiments, the lumen has a circular or oval cross-sectional shape of constant area throughout the length of the lumen with a diameter or largest transverse dimension of about 8 mm.

In some embodiments, the length of the tube portion is about 300 mm and the diameter of lumen is about 8 mm. In other embodiments, the length of the tube portion is about 240 mm and the diameter of lumen is about 6 mm.

Again referring to FIGS. 1 and 2, the tube portion 16 also comprises one or more bite-resistant zones 22 (one shown) located intermediately along the tube portion 16 between the proximal end 12 and the distal end 14 of the endotracheal tube 10.

A bite-resistant zone usually is located to be positioned during normal use between the upper and lower teeth, for example, in proximity to the upper and lower incisors. When so positioned, conventional endotracheal tubes are vulnerable to partial or complete occlusion or, possibly trans-section, caused by patient bite-down on the tube. Bite-down inadvertently can occur during light anesthesia or during emergence from general anesthesia. Airway obstruction associated with complete or partial occlusion of an endotracheal tube can result in hypoxemia. In young muscular patients, pulmonary edema may also develop, induced by negative pressures generated during attempts at inspiration.

The masseter muscles, which are the muscles employed to exert biting forces, can generate significant pressures on an object located between the teeth. For example, males can exert a biting force in the incisor region of up to about 18 kg (177 N) and females can exert up to about 11 kg (108 N). Higher forces in a range of from about 25 kg (245 N) to about 130 kg (1275 N) can be exerted by other teeth, with some individuals exerting bite forces that are a hundred or more kilograms higher. Examples of typical bite forces are: from about 10 kg (98 N) to about 15 kg (147 N) for the incisors; from about 30 kg (294 N) to about 50 kg (490 N) for the canines; from about 40 kg (392 N) to about 60 kg (589 N) for the premolars; from about 30 kg (294 N) to about 40 kg (392 N) for the first molars; and from about 50 kg (490 N) to about 75 kg (736 N) for the second molars.

As shown in FIGS. 1 and 2, to resist closure or obstruction resulting from bite-down, the tube portion 16 can comprise in the bite-resistant zone 22 a reinforcement member 24 having any one of a variety of structures or configurations. For example, the reinforcement member can be resilient and can be formed largely or entirely of wire. As illustrated in FIGS. 1 and 2, the reinforcement member 24 comprises a set of multiple closely-spaced turns of resilient wire 26 extending around the lumen 18 in a helical coil in the bite-resistant zone 22, which wires are configured to resist biting. In some embodiments, the combination of the tube wall and the closely-spaced wire turns in the bite-resistant zone can simulate a solid pipe. In certain embodiments, two side-by-side lengths of wire are coiled together into intercalated helices. Alternatively, a reinforcement member can comprise multiple discontinuous sections of wire. Other suitable structures or configurations of the reinforcement member will be known or apparent to a person of ordinary skill in the art, in light of this disclosure, or will become known or apparent in the future, as the art develops.

The regions of tube portion outside the bite-resistant zone can be free of the reinforcement member, for example, to permit the tube portion substantial flexibility. If desired, as is further described herein, the regions of the tube portion outside the bite-resistant zone can be provided with limited reinforcement, for example, a kink-inhibiting structure, which can permit significant flexing of the tube portion but prevent kinking.

A reinforcement member can desirably function to keep the lumen open during biting. That is, the reinforcement member can maintain patency of the lumen and permit the ventilating gas to flow through the lumen during biting. For example, the reinforcement member can have adequate strength or other structural properties to resist closure of the lumen during a bite-down event, when a patient may exert an unusual biting force. The reinforcement member also can have adequate resiliency to return to its or near to its original shape and/or position.

The closely-spaced wire turns of a reinforcement member can have any suitable spacing between the turns which provides adequate reinforcement to prevent closure of the lumen. For example, the spacing can be less than the thickness of the wire employed to form the wire turns. In some embodiments, the spacing can be from about 0.1 mm to about 1.0 mm. In certain embodiments, there can be close adjacency between the wire turns, or contact between adjacent wire turns.

In certain embodiments of endotracheal tubes, the closely-spaced wire turns can have any desired extent along the tube portion that is consistent with the objectives described herein. In some embodiments, the bite-resistant structure can have a length sufficient to accommodate a range of distances along the tube portion where the incisor teeth could potentially cause occlusion of the lumen during biting, for example, an extent in the range of from about 5 mm to about 100 mm, or in the range of from about 20 mm to about 60 mm. In some embodiments, the closely-spaced wire turns have an extent of about 40 mm along the tube portion. The extent of closely-spaced wire turns along the tube portion can comprise any desired proportion of the length of the tube portion, but typically is from about 5 percent to about 40 percent of the length of the tube portion, or from about 10 percent to about 20 percent of the length of the tube portion so as to provide sufficient flexibility towards one or both of the distal end and the proximal end. Other suitable bite-resistant structures can have a similar extent.

A reinforcement member can have various constructions. For example, the reinforcement member can comprise continuous, woven, braided and/or interlaced wire. Also, the reinforcement member can be configured as a cylinder, the cylinder having a circular, oval, elliptical or “C”-shaped cross-section. A C-shaped cross-section can provide a channel to accommodate an air passage, as is further described herein, or another desired passage or conduit.

Further, the reinforcement member can comprise one or more resilient wire members having a sinuous, zigzag or other fluctuating configuration, the fluctuation being with respect to a circumferential or helical line extending around the tube portion.

A reinforcement member also can comprise one or more strands or filaments of a metallic material or of a composite material or of a carbon fiber material or of a suitable polymeric material, for example, a polymeric material having a relatively high tensile strength and a relatively low elasticity. Still further, a reinforcement member can employ a material in a woven, a braided or an interlaced configuration, if desired, and optionally, as is described elsewhere herein, can be completely or partially embedded in the tube wall.

A tube portion can be flexible about one or more axes transverse to the major axis of the tube portion, which is to say transverse to the direction in which the lumen extends. The tube portion can be flexible distally of the bite-resistant zone, which flexibility can help in properly positioning the endotracheal tube within the patient. Alternatively, or in addition, the tube portion can be flexible proximally of the bite-resistant zone, which flexibility can help in positioning the proximal end of the endotracheal tube for connection with an external service, for example, a source of ventilating gas. The flexibility can be in the entire extent of the tube portion proximally or distally of the bite-resistant zone, or can be in one or more limited regions.

However, in some cases, a flexible tube portion may kink undesirably, restricting or preventing the flow of ventilating gas through the lumen. Accordingly, in such cases, the reinforcement member can comprise additional reinforcement, for example, a kink-inhibiting structure or structures, located distally and/or proximally of the bite-resistant structure, which additional reinforcement permits flexing of the tube portion yet provides limited support or strengthening to resist kinking. The additional reinforcement(s), for example, kink-inhibiting structure(s), each can be independent of or contiguous with the reinforcement member or members. The reinforcement member and additional reinforcement also can be present as one structure, for example, as a one-piece design.

As shown in FIGS. 1 and 2, a kink-inhibiting structure can comprise two sets of widely-spaced turns of wire outside the bite-resistant zone 22. The turns of a kink-inhibiting structure can extend a sufficient distance along the tube portion from the bite-resistant zone to alleviate or prevent kinking of the tube portion at the respective end(s) of the bite-resistant zone. A sufficient distance can be the entire available length of the tube portion or a fractional portion thereof, for example, from about 10 percent to about 80 percent, from about 30 percent to 60 percent of the available length of the tube portion.

Any suitable spacing can be employed between the widely-spaced turns of the additional wire reinforcement of a kink-inhibiting structure provided adequate reinforcement is present to prevent closure of the lumen. For example, the spacing between the widely-spaced turns can be at least the thickness of the wire, a spacing in the range of from about 0.5 mm to about 10 mm, a spacing in the range of from about 3 mm to about 8 mm, or a spacing in the range of from about 4 mm to about 6 mm.

Other suitable configurations of a kink-inhibiting structure will be known or apparent to a person of ordinary skill in the art in light of this disclosure, or will become known or apparent in the future as the art develops. It should be understood that certain embodiments of oropharyngeal devices described herein lack a kink-inhibiting structure.

As shown in FIG. 1, the reinforcement member 24 of the illustrated endotracheal tube 10, comprising both closely-spaced wire turns 26 and widely-spaced wire turns 28 (kink-inhibiting structures), extends along a minor portion of the length of the endotracheal tube 10. Such a structure, where the reinforcement member and the kink-inhibiting structures, if present, occupy a minor proportion of the length of an endotracheal tube, can provide substantial regions of tube portion that lack reinforcement and therefore, are free to flex, permitting manipulation of endotracheal tube into a desired configuration. For example, a reinforcement member can extend along an endotracheal tube for a distance in the range of from about 10 percent to about 50 percent of the length of the endotracheal tube, which distance can be in the range of from about 20 percent to about 35 percent of the length of the endotracheal tube. Without additional reinforcement present throughout the tube portion, the tube wall should be of sufficient thickness and/or construct to resist kinking. Other reinforcement structures, if employed in place of closely-spaced (and/or widely-spaced) wire reinforcement turns, can be similarly disposed.

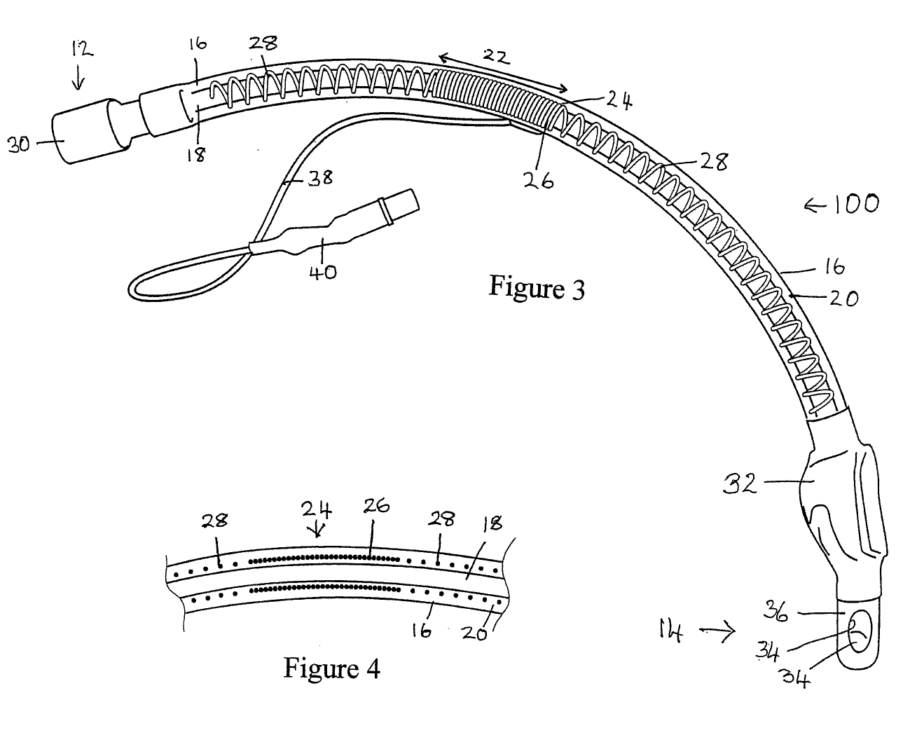

The endotracheal tube 100 illustrated in FIGS. 3 and 4 is generally similar to the endotracheal tube shown in FIGS. 1 and 2, with similar parts being similarly referenced. A major difference is that in the embodiment shown in FIGS. 3 and 4, the reinforcement member 24, which includes closely-spaced wire turns 26 and widely-spaced wire turns 28, extends throughout a major portion of the length of the endotracheal tube 100. For example, the reinforcement member can occupy from about 60 to about 100 percent of the length of the endotracheal tube between end fittings. As illustrated in FIG. 3, the reinforcement member 24 occupies more or less the entire available length of the endotracheal tube 100 between end fittings, which are a female-adaptor 30 and a male adaptor 40.

Returning to the cross-sectional view in FIG. 2, the reinforcement member 24 is located within the outer periphery of and embedded within the tube wall 20. No wire, wire end, or wire fragment is exposed internally or externally of the endotracheal tube. However, other arrangements are possible, for example, where a part of a reinforcement member and/or a kink-inhibiting structure is exposed internally or externally of the tube portion.

In some embodiments of endotracheal tubes (not shown), the reinforcement member is located outside the outer periphery of the tube wall, and optionally is disposed entirely outside the tube wall. For example, the reinforcement member can be coiled around the outer periphery of the tube portion. In certain embodiments of endotracheal tubes (not shown), the reinforcement member and/or kink-inhibiting structure are located within a lumen, and optionally are disposed entirely within the lumen. In embodiments where the reinforcement member and/or kink-inhibiting structure are located outside the tube wall or in the lumen, the reinforcement member and kink-inhibiting structure and the tube portion can be a tight fit, for example, one within the other with substantial contacting surfaces, to enhance resistance to biting or other lumen-closing forces.

The reinforcement member and kink-inhibiting structure(s), if present, can comprise a single coiled length of wire providing both closely-spaced wire turns and widely-spaced wire turns, i.e., a bite-resistant and kink-inhibiting structure. For example, a single length of wire can be variably coiled according to location along the endotracheal tube, being relatively tightly coiled in the bite-resistant zone to provide closely-spaced wire turns and being relatively loosely coiled distally and/or proximally of the bite-resistant zone to provide a kink-inhibiting structure of widely-spaced turns at one or both ends of the closely-spaced turns.

In some embodiments, the closely-spaced wire turns are provided by one length of wire and each of the widely-spaced wire turns, if present, are provided by an additional length of wire, for example, two additional lengths of wire being employed if two sets of widely-spaced wire turns are present. In certain embodiments, one or more of the wire turns can be formed by a plurality of pieces of wire or the like, and can be arranged to be contiguous or non-contiguous with one another. The set or sets of widely-spaced wire turns can be contiguous with or adjacent to the set of closely-spaced wire turns or can be spaced apart from the set of closely-spaced wire turns.

In some embodiments, the tube portion comprises a plurality of reinforcement members located in a plurality of bite-resistant zones, for example, for use with patients of different anatomies or for different degrees of insertion of the endotracheal tube into the patient. In certain embodiments, a single reinforcement member can extend into such a plurality of bite-resistant zones. In particular embodiments, the tube portion comprises a plurality of kink-inhibiting structures.

A reinforcement member can be positioned at any suitable distance along an endotracheal tube depending on its intended application. For example, referring to an endotracheal tube having a tube portion with a length of about 300 mm, the center point of a bite-resistant zone of a reinforcement member can be positioned at a distance in the range of from about 190 mm to about 230 mm from the distal end of the tube portion. The reinforcement member can extend throughout the entire length of the tube portion, for example, the reinforcement member can comprise widely-spaced wire turns extending a distance of about 190 mm along the tube portion from the distal end and from about 230 mm along the tube portion from the distal end to the proximal end with closely-spaced wire turns extending from about the 190 mm mark to about the 230 mm mark.

A bite-resistant zone can be robust. For example, the bite-resistant zone can be sufficiently robust to resist a likely maximum biting force exerted by an intended user without closing the lumen to the flow of oxygen, air or other gas in an unacceptable manner. The maximum likely biting force can be predetermined according to available medical and anatomical information. Accordingly, the bite-resistant zone can be sufficiently robust so that when the maximum likely biting force is exerted, the lumen retains sufficient patency to maintain the vitality of gas flow through the lumen.

In some embodiments, a bite-resistant zone can resist a biting force selected from the group consisting of at least about 25 kg/cm², at least about 100 kg/cm², and at least about 500 kg/cm², while permitting an adequate flow of ventilating gas through the lumen. The biting pressure resistance of a bite-resistant zone can be selected based on the intended patient. For example, an endotracheal tube intended for use with a healthy adult male human can be constructed to resist a biting pressure of about 500 kg/cm², while an endotracheal tube intended for a healthy adult female human, or a child, can be constructed to resist a biting pressure of about 100 kg/cm². The bite-resistance performance of an endotracheal tube can be determined experimentally, if desired.

As an alternative or supplement to an actual bite-resistance performance characteristic, an endotracheal tube, or other device using the concepts disclosed herein, can have a rating indicating a probable bite-resistance capacity of the tube. The rating can be calculated, or otherwise determined, based upon the construction of the endotracheal tube and the materials employed.

A bite-resistant zone can be given suitable robustness by employing wire of a suitable thickness and material and by a relatively close packing of the turns of wire. For example, a bite-resistant zone can be constructed so that the teeth of an intended patient generally cannot separate and be interposed between adjacent turns of wire.

A reinforcement member can be formed of any suitable material capable of providing a desired level of bite-resistance when suitably configured, for example, configured as side-by-side, closely-spaced turns extending around part or the entire circumference of a lumen, or configured in another suitable manner. For example, a reinforcement member can comprise a metal, a metal alloy such as stainless steel or nickel titanium (e.g., Nitinol), carbon fibers, other composite materials, and combinations thereof.

One suitable material for a reinforcement member comprises a high-strength steel alloy, for example, a steel alloy meeting ASTM standard A514 or A517. Other suitable materials include: a tempered high-carbon steel, for example, a high-carbon steel meeting ASTM standard A228; a cold-rolled stainless steel, for example, a cold-rolled stainless steel meeting American Iron and Steel Institute (AISI) standard AISI 302; and a titanium alloy.

A wire employed as a reinforcement member can have any suitable diameter. More specifically, the diameter can be selected according to the size and application of the endotracheal tube. For example, suitable wire can have a U.S. Steel Wire Gauge in the range of from about gauge 20 (0.035 in, 0.89 mm) to about gauge 28 (0.016 in, 0.41 mm). More specific examples of wire include those which are gauge 22 (0.0286 in, 0.73 mm), gauge 23 (0.0258 in, 0.66 mm), gauge 24 (0.023 in, 0.58 mm), and gauge 25 (0.0204 in, 0.52 mm). (The approximate wire diameter for each gauge size is given in parenthesis.) All references herein to a wire gauge size refer to U.S. steel wire gauge, unless otherwise indicated.

A reinforcement member can comprise a wire having suitable mechanical characteristics such as diameter, tensile strength, and elastic modulus. For example, the wire can have a tensile strength of from about 500 MPa to about 1000 MPa (about 50 Kpsi to about 150 Kpsi where “kpsi” is kilo pounds per square inch). Also, the wire material can have an elastic modulus of from about 20 million psi (about 0.14 MPa) to about 60 million psi (about 0.41 MPa).

Accordingly, the principles and concepts taught herein provide an adaptable system for furnishing a variety of bite-resistant structures having different performance characteristics suitable for different individuals and uses. For example, by varying the gauge of wire employed for the wire turns, and the spacing between the wire turns, the resistance to biting provided by the reinforcement member can be adjusted for different diameter tubes and to suit different individuals.

Although unnecessary, the bite-receiving zone can yield during biting to resist bite-induced injury while maintaining patency of the lumen. To this end, the bite-resistant zone can be resiliently yieldable, or provide cushioning, to receive or absorb the forces induced during bite-down without inflicting dental or other damage, or possibly pain, on the patient.

Resilient yieldability can be provided by using closely-spaced wire turns of a metal material, for example, a metal alloy wire such as a stainless steel wire or a nickel-titanium (Nitinol) wire. Resilient yieldability or cushioning also can be provided by appropriate construction of and selection of materials for the tube wall. For example, resilient yieldability or cushioning can be provided by employing appropriate materials of construction and/or an adequately thick tube wall in the bite-resistant zone, which tube wall can be thicker in the bite-resistant zone than elsewhere.

In some embodiments, the tube portion is flexible distally of the bite-resistant zone to enable appropriate positioning of the distal end of the endotracheal tube in the patient. In certain embodiments, distal flexibility is not required. Optionally, the tube portion can be flexible proximally of the bite-resistant zone, for example, to facilitate connection with a source of ventilating gas. In some embodiments, the tube portion can be flexible about one or more axes transverse to the major axis of the tube portion.

The bite-resistant zone also can be relatively inflexible about axes transverse to the major axis of the tube portion or can have limited flexibility about those axes. Substantial flexibility may reduce the bite-resistant qualities of the bite-resistant zone. Accordingly, certain embodiments comprise an endotracheal tube having little flexibility in the bite-resistant zone and relatively high resistance to biting. In other embodiments, an endotracheal tube can be relatively flexible in the bite-resistant zone and have relatively low resistance to biting but remain capable of maintaining patency of the lumen during biting because of the particular design and/or materials of construction.

Returning to FIG. 1, the proximal end 12 of endotracheal tube 10 comprises a female adaptor 30 for connecting with a source of ventilating gas, a breathing circuit, or another suitable service, for example, suction, irrigation or an anesthetic or other biochemically active treatment. A female adaptor as shown comprises a simple ring connector which terminates the proximal end of the tube portion of the endotracheal tube. However, the female adaptor can have any suitably more or less elaborate configuration or structure according to the intended use of the proximal end of the endotracheal tube, as will be apparent to a person of ordinary skill in the art.

Depending upon its intended application, the distal end of an endotracheal tube can be configured to communicate ventilating gas into the trachea of a patient or to perform another useful function. For example, as shown in FIG. 1, the endotracheal tube 10 can comprise, at the distal end 14, a balloon cuff 32 extending externally around the tube portion 16, and one or more gas delivery ports 34 at the distal tip 36 of the endotracheal tube 10. The one or more gas delivery ports 34 can be formed in the tube portion 16 or may be provided in a separate fitting which is assembled to the tube portion 16.

The balloon cuff can be configured to occlude the patient's trachea, preventing air or gas leaks and ingress of solids or liquids into the patient's airway while permitting ventilating gas to flow from lumen, which extends through the balloon cuff, through the port(s) into the patient's trachea, permitting aspiration. Optionally, the balloon cuff can comprise a ring-shaped inflatable balloon extending around the tube portion.

Again referring to FIG. 1, the balloon cuff 32 can be supplied with air or other inflation gas via an air channel 38, which is connected to an external oxygen, air or gas supply, or a manual pump, or other air or gas source, via a male adaptor 40. The air channel 38 can be an independent flexible tube coupled to the balloon cuff 32 as is shown. The air channel also can be integrated into, or mounted on, the tube wall of the tube portion of the endotracheal tube. For example, the wire turns or other reinforcement member(s) can be formed with a longitudinal gap with an air channel located in the gap, thereby providing a compact construction. The balloon cuff can be configured to be effectively inflated with low-pressure air or gas. In use, the balloon cuff can be placed within the trachea beyond the vocal cords, or in another suitable location.

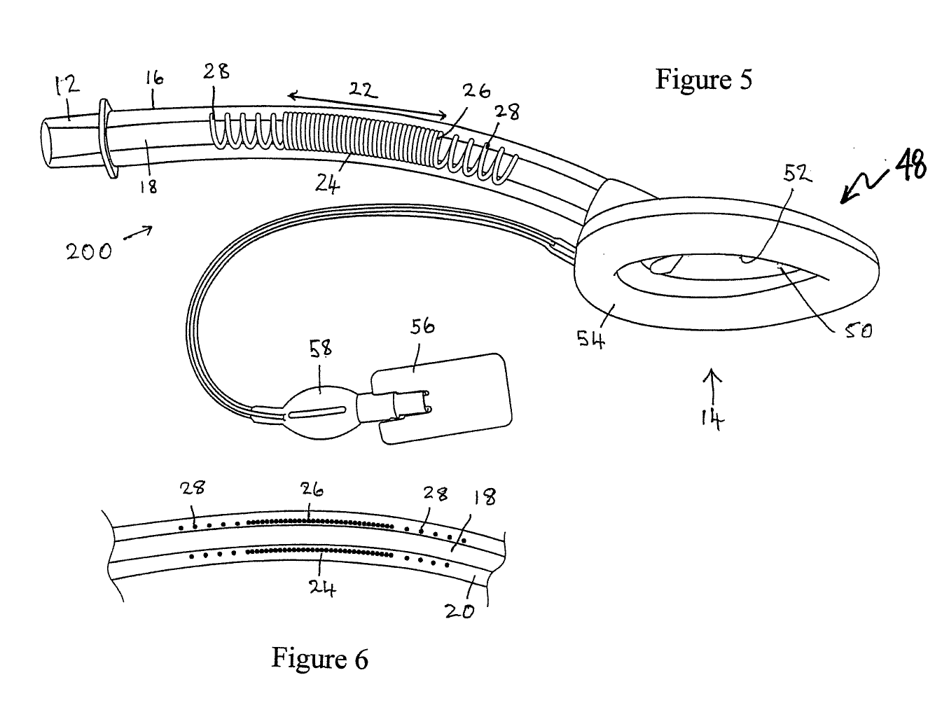

Another type of oropharyngeal device, a laryngeal mask airway, is illustrated in FIGS. 5 and 6. The exemplary laryngeal mask airway generally has structural similarity to the exemplary endotracheal tube shown in FIGS. 1 and 2, with major differences being that the laryngeal mask airway is shorter than the endotracheal tube and that the laryngeal mask airway has a different fitting at its distal end. Similar features or parts are referenced with the same reference numerals as are used in FIGS. 1 and 2 and thus, will not be described again in detail as the same description applies equally to these features in a laryngeal mask airway.

As shown in FIGS. 5 and 6, a laryngeal mask airway 200 comprises a proximal end 12, a distal end 14, a tube portion 16 having a lumen 18 and a tube wall 20, as well as a bite-resistant zone 22, a reinforcement member 24 comprising closely-spaced wire turns 26 and widely-spaced wire turns 28 (kink-inhibiting structures), and a female adaptor 30.

The tube portion provides what is sometimes called the “stem” of the laryngeal mask airway and can have any suitable dimensions and configuration. For example, the tube portion can have an approximately circular cross-sectional shape with a diameter in the range of from about 6 mm to about 25 mm, or from about 10 mm to about 16 mm. The tube portion can have a cross-sectional shape such as oval or approximately rectangular shape, which cross-sectional shape can be of comparable area to the circular configuration, if desired. Also, the laryngeal mask airway can have a length, measured along the tube portion, of from about 60 mm to about 200 mm or from about 80 mm to about 120 mm, or any other suitable length.

In some cases, laryngeal mask airways can have a major transverse dimension which is greater than that of an endotracheal tube intended for the same patient.

The tube wall of a tube portion can have a thickness greater than 0.5 mm, for example, from about 0.5 mm to about 10 mm, or from about 1 mm to about 4 mm.

In a laryngeal mask airway, a reinforcement member, for example, closely-spaced wire turns, can have any desired extent along the tube portion, such as an extent in the range of from about 10 mm to about 150 mm, or in the range of from about 40 mm to about 100 mm. In certain embodiments, the closely-spaced wire turns have an extent of about 70 mm along the tube portion. The extent of closely-spaced wire turns along the tube portion can comprise any desired proportion of the length of the tube portion, for example, from about 10 percent to about 60 percent of the length of the tube portion or from about 25 percent to about 40 percent of the length of the tube portion. Other suitable bite-resistant structures can have a similar extent. Nevertheless, the extent of the reinforcement member (and the bite-resistant zone) needs to be balanced with the flexibility required towards the distal and proximal ends of the tube portion while using the device.

As with the endotracheal tube, the reinforcement member can be positioned at any suitable distance along a laryngeal mask airway depending on its intended application, including the specific examples recited for an endotracheal tube.

As shown in FIG. 5, at its distal end 14, instead of a balloon cuff and a ported distal tip, the laryngeal mask airway 200 is provided with a laryngeal mask 48. The laryngeal mask 48 comprises a bowl fitting 50, which communicates with the tube portion 16 of the laryngeal mask airway 200 and terminates the laryngeal mask airway 200. The bowl fitting 50 comprises a wide aperture 52 providing relatively high volume gas flow capabilities and can be surrounded by an inflatable cuff 54. As shown, a hand-operable pump 56, which comprises a resilient bulb 58, is coupled with and in fluid communication with the inflatable cuff 54 to provide inflation air or other gas. The pump optionally can be operable to deflate the cuff to facilitate intubation of the laryngeal mask airway into a patient. The laryngeal mask can be configured to extend over the larynx of the patient and communicate ventilating gas to the pharyngeal airway of the patient. Other suitable cuff inflation and/or deflation devices, or systems, will be known or apparent to a person of ordinary skill in the art, in light of this disclosure, or will become known or apparent in the future, as the art develops.

A laryngeal mask airway can be relatively shorter than a corresponding endotracheal tube for a given patient so as to reach less deeply into the patient's respiratory tract. Accordingly, a reinforcement member can occupy a major portion of the length of a tube portion of a laryngeal mask airway, while desirably leaving portions of the tube portion free of reinforcement to permit flexing of the tube portion. For example, in the embodiment of the laryngeal mask airway illustrated in FIGS. 5 and 6, relatively short lengths of widely-spaced turns 28 are employed to leave adequate unreinforced regions of the tube portion 16 towards the distal end 14 and the proximal end 12 to provide desired flexibility.

A wire employed as a reinforcement member for a laryngeal mask airway can have any suitable diameter, for example, those described above for an endotracheal tube. More specifically, the wire can have a diameter selected according to the size of the laryngeal mask airway and its intended application. For example, suitable wire can have a U.S. Steel Wire Gauge in the range of from about gauge 17 (0.054 in, 1.37 mm) to about gauge 23 (0.0258 in, 0.66 mm). Other more specific examples include gauge 19 (0.0286 in, 0.73 mm), gauge 20 (0.035 in, 0.89 mm), and gauge 21 (0.032 in, 0.81 mm). (The approximate wire diameter for each gauge size is given in parenthesis.)

Any suitable manufacturing process can be employed to make an oropharyngeal device as described herein such as an endotracheal tube or a laryngeal mask airway. For example, in some embodiments, the manufacturing process can comprise preforming, preassembling or predisposing a reinforcement member in an appropriate manner, for example, forming a structure suitable for disposition in a bite-resistant zone; and building a tube portion outside, inside or around the reinforcement member, by extrusion, by molding, by deposition, or by another suitable manner to create an orolaryngeal device comprising a lumen, wherein the reinforcement member surrounds the lumen and the oropharyngeal device is capable of maintaining the patency of a lumen disposed within the orolaryngeal device during a biting by a patient. One or more distal end components, proximal end components, and any other components of the orolaryngeal device then can be assembled with the reinforced tube portion. An exemplary description of a manufacturing process for making an oropharyngeal device, which has a composite tube portion comprising concentric inner and outer tubular layers, follows.

In one step, an inner layer of the composite tube portion is extruded from a polymeric material to have a suitable wall thickness, for example, a thickness of from about 1 mm to about 5 mm. The inner composite tube layer is formed with a smooth inner surface or lining to promote laminar low-resistance airflow. The inner tube layer optionally is treated, coated, or otherwise provided with a lining to reduce airflow resistance.

In another step, a suitable reinforcement member, for example, one or more lengths of wire, are wrapped around the inner tube layer at the desired location along the length of the tube portion and in any desired configuration, for example, a helical coil, or another suitable pattern such as sinuous, zigzag, continuous, interrupted or a configuration which fluctuates either side of a circumferential or helical line extending around the inner tube portion.

In a further step, the assembly of the reinforcement member and the inner tube layer is placed within a mold, which defines the desired outer geometry of the outer tube portion. The mold is coated or filled with a polymeric material to form the outer tube layer of the oropharyngeal device around the reinforcement member and the inner tube layer.

An air channel, if employed, is embedded in the outer tube layer or is attached to the tube portion, or otherwise is coupled with an inflatable cuff subsequently to molding. For compactness, the wire turns or other reinforcement member is formed with a longitudinal gap and the air channel is inserted in the gap during manufacture, with the outer tube layer formed over or around the air channel.

Once completed, the proximal end of the tube portion is capped with a suitable adaptor such as a female adaptor, to enable the end product to be coupled with a breathing circuit or other suitable ventilation source, or other service. An appropriate distal termination component is added to the tube portion, before or after assembly of the proximal end adaptor. The distal termination structure and functionalization is selected according to the intended end use of the oropharyngeal device being fabricated.

For example, the distal termination can comprise an apertured distal tip or the like, and optionally, a balloon cuff or other suitable sealing device can be employed to provide an endotracheal tube such as shown in FIGS. 1 and 3. Alternatively, to provide a laryngeal mask airway such as the laryngeal mask airway illustrated in FIG. 5, the distal termination can comprise one or more laryngeal mask components such as an apertured bowl fitting, and if desired, an inflatable cuff and a pump or air source connector for inflating the same. Other suitable distal terminations which can be employed to provide other desired oropharyngeal devices will be known or apparent to a person of ordinary skill in the art, in light of this disclosure, or will become known or apparent in the future, as the art develops.

In certain embodiments, a manufacturing process for making an oropharyngeal device as described herein can include retrofitting a reinforcement member such as a coiled wire to an existing oropharyngeal device to provide resistance to bite-down. Such a reinforcement member which is retrofitted can comprise a plurality of closely-spaced wire turns and a plurality of widely-spaced wire turns in one piece, or as multiple pieces, or as an assembly of multiple pieces, or can comprise an equivalent structure to the reinforcement member, as is described herein or will be known or apparent to a person of ordinary skill in the art, in light of this disclosure, or will become known or apparent in the future, as the art develops. A reinforcement member can be retrofitted externally or internally to a suitable oropharyngeal device in an appropriate bite-resistant zone.

In various embodiments, the manufacturing process can include wrapping, positioning or placing a reinforcement member to support a pre-existing lumen, for example, wrapping appropriately a wire around the lumen, to provide a bite-resistant zone. Such embodiments also can include wrapping, positioning or placing a kink-inhibiting structure or structures to support the pre-existing lumen, for example, wrapping appropriately a wire around the lumen. In some embodiments, if the reinforcement member protrudes or is visible outside the tube portion, all or a portion of the reinforcement member can be coated or covered with an appropriate material to protect the reinforcement member and/or to render the device suitable for inserting in a patient.

An exemplary use of an endotracheal tube will now be described. A female adaptor at the proximal end of an endotracheal tube is coupled with a breathing apparatus. The distal end of the endotracheal tube is inserted through the mouth of a patient, past the patient's trachea, with a bite-resistant zone located between the patient's upper and lower dentition. Once so positioned, a balloon cuff is inflated to form a seal with the interior lining of the trachea. After the seal is established, the breathing apparatus is operated to apply positive breathing gas pressure to the proximal end to ventilate the patient's lungs. The seal formed between the balloon cuff and the inner lining of the trachea can help prevent aspiration into the patient's lungs of solids, such as material regurgitated from the stomach.

During bite-down, with an endotracheal tube appropriately positioned, the patient's teeth or other dentition should clamp down on to the reinforcement member in the bite-resistant zone. Here, the metal wire or other reinforcement member can resist the biting forces and maintain patency of the lumen, permitting breathing gas to flow through the lumen to the patient. In certain embodiments, the tube portion and the resilient reinforcement member can yield a little to accommodate the load, while keeping the lumen open to provide an adequate gas flow rate. By appropriate selection of parameters of the reinforcement member, for example, the diameter, the tensile strength and the elastic modulus of the closely-spaced wire turns, the reinforcement member can have sufficient resilient strength to maintain adequate patency of the lumen during a severe bite-down such as can be imposed by a young adult male coming out of general anesthesia.

The reinforcement member and the bite-resistant zone can have a sufficient extent along the tube portion as to be aligned between the patient's teeth in a variety of positions of the endotracheal tube in the patient's mouth. Such positions can include positions where the tube portion extends from the front of the mouth, the side of the mouth, between the incisors, between the canines, or between the molars. Also, by providing a reinforcement member and a bite-resistant zone with adequate lengths, different oropharyngeal anatomies of different patients can be accommodated with one device, i.e., the distal end of the endotracheal tube can be properly positioned while maintaining the bite-resistant zone between the patient's teeth.

As the endotracheal tube is manipulated during intubation and ventilation, the unreinforced regions of the tube portion can permit substantial flexing of the tube portion about one or more transverse axes, facilitating intubation and connection to the breathing apparatus or other service. Such flexing can also help accommodate patient movements, such as head turns, without dislodging the endotracheal tube.

The use of a laryngeal mask airway is generally similar to the use of an endotracheal tube with regard to the positioning and function of a reinforcement member and the connection of the proximal end to a breathing apparatus or other useful system. However, a laryngeal mask airway is intubated into the patient's respiratory tract in a different manner as will be known or apparent to a person of ordinary skill in the art.

For example, by way of preparation for intubation, an inflatable cuff of a laryngeal mask airway is deflated, and the laryngeal mask airway is externally lubricated, if desired. Also, the patient can be sedated. The distal tip of the laryngeal mask airway then is inserted into the mouth of the patient, over the tongue and advanced into the pharynx while pressing the distal tip against the patient's hard palate. The laryngeal mask airway is inserted into the hypopharynx and the tip of the inflatable cuff is positioned at the upper esophageal sphincter. Once the inflatable cuff is properly positioned, it is inflated to create a seal. During use of the laryngeal mask airway, a reinforcement member can function as described for a reinforcement member of an endotracheal tube, for example, to resist closure of the lumen and permit flow of oxygen, air or gas through the lumen.

Oropharyngeal devices and methods as described herein can be employed in the treatment of mammals, including in particular, humans, to facilitate ventilation of a subject during surgery or a medical crisis, or for other purposes. Furthermore, oropharyngeal devices and methods as described herein can be employed for veterinary purposes for treatment of non-human mammals including, for example, horses, cattle, sheep, llamas, husbanded animals, pets, dogs, cats, laboratory animals, mice, rats, primates, apes, monkeys, endangered species, animals employed for sports, breeding, entertainment, law enforcement, draft usage, zoological or other purposes.

The entire disclosure of each and every patent and non-patent publication, is incorporated by reference herein in its entirety for all purposes. Should there be a conflict between the meaning of a term employed in this specification and the usage of that term in material incorporated by reference from another publication, the meaning as used herein is intended to prevail.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art.

The terms “include,” “includes,” “including,” have,” “has,” “having,” “contain,” “contains,” and “containing” are to be understood as being open-ended and not to exclude additional, unrecited elements or method steps unless specifically stated otherwise.

Throughout the description, where compositions, instruments, devices, apparatus, systems, or processes are described as having, including, or comprising specific components or elements, and in the case of processes, having, including, or comprising specific steps, it is contemplated that compositions, instruments, devices, apparatus, systems, and processes can also consist essentially of, or consist of, the recited components, elements or process steps.

In the application, where an element or component is said to be included in and/or selected from a list or group of recited elements or components, it should be understood that the element or component can be any one of the recited elements or components or can be selected from a group consisting of two or more of the recited elements or components.

The use of the singular herein is intended to include the plural (and vice versa) unless specifically stated otherwise. In addition, where the term “about” is used before a quantitative value, the specific quantitative value itself is intended to be included, unless specifically stated otherwise.

It is to be understood that the order of steps or order for performing certain actions is immaterial so long as the described process remains operable. Moreover, two or more steps or actions may be conducted simultaneously, unless the context indicates otherwise. In addition, any proportions recited herein are to be understood to be proportions by weight, based upon the weight of the relevant composition, unless the context indicates otherwise.

The description of embodiments and examples herein is to be understood as including combinations of the various features and elements of the invention, and of their disclosed or suggested alternatives, including alternatives disclosed, implied or suggested in any one or more of the various methods, products, compositions, systems, apparatus, instruments, aspects, embodiments, and examples described in the specification and drawings, and to include any other written or illustrated combination or grouping of elements or of the possible practice of the invention, except for groups or combinations of elements that are incompatible with, or contrary to the purposes of the invention, as will be or become apparent to a person of ordinary skill. Further, discussion herein of the features, characteristics, manufacture, and use of one oropharyngeal device, for example, an endotracheal tube, apply equally to other oropharyngeal devices, for example, a laryngeal mask airway, unless otherwise stated or understood.

The present application and its teachings encompass embodiments in other specific forms without departing from the spirit or essential characteristics thereof. The foregoing embodiments are therefore to be considered in all respects illustrative rather than limiting on the present teachings described herein. The scope of the present invention is thus indicated by the appended claims rather than by the foregoing description, and all changes that come within the meaning and range of equivalency of the claims are intended to be embraced therein.

1. An oropharyngeal device for use in ventilating a patient by supplying a ventilating gas to an airway of the patient, the oropharyngeal device comprising:
a proximal end connectable with a source of ventilating gas;
a distal end disposable within the airway of the patient with the proximal end located externally of the patient; and
a tube portion extending between the proximal end and the distal end, the tube portion defining a lumen permitting ventilating gas to flow through the oropharyngeal device from the proximal end to the distal end and comprising:
a tube wall comprising a flexible resilient polymeric material; and
a resilient wire reinforcement extending in multiple turns around the lumen in a bite-resistant zone, the bite-resistant zone being located intermediately along the tube portion between the proximal end and the distal end, and being subject to engagement between the teeth of the patient during use;
wherein the turns of the resilient wire reinforcement are spaced closely together to resist biting pressure and keep the lumen open during biting to permit the ventilating gas to flow through the lumen.

2. An oropharyngeal device according to claim 1 wherein the turns of the resilient wire reinforcement have a spacing between the turns selected from the group consisting of: less than the thickness of the wire; from about 0.1 to about 1.0 mm; close adjacency between the turns; and contact between adjacent turns.

3. An oropharyngeal device according to claim 1 wherein the resilient wire reinforcement comprises widely-spaced wire turns outside the bite-resistant zone and adjacent one or both ends of the closely-spaced wire turns.

4. An oropharyngeal device according to claim 3 comprising a spacing between the widely-spaced wire turns selected from the group consisting of at least the thickness of the wire, a spacing in the range of from about 0.5 mm to about 10 mm, a spacing in the range of from about 3 mm to about 8 mm, and a spacing in the range of from about 4 mm to about 6 mm.

5. An oropharyngeal device according to claim 1 wherein the resilient wire reinforcement comprises a continuous length of wire.

6. An oropharyngeal device according to claim 1 wherein the resilient wire reinforcement comprises multiple discontinuous sections of wire.

7. An oropharyngeal device according to claim 1 wherein the resilient wire reinforcement is located within the outer periphery of the tube wall.

8. An oropharyngeal device according to claim 1 wherein the resilient wire reinforcement is embedded within the tube wall and no wire is exposed internally or externally of the oropharyngeal device.

9. An oropharyngeal device according to claim 1 wherein the resilient wire reinforcement is located outside the outer periphery of the tube wall.

10. An oropharyngeal device according to claim 1 wherein the resilient wire reinforcement comprises a coiled length of wire, the wire being variably coiled according to location along the oropharyngeal device, being relatively tightly-coiled in the bite-resistant zone and relatively loosely-coiled distally and proximally of the bite-resistant zone.

11. An oropharyngeal device according to claim 1 wherein the bite-resistant zone can resist a biting pressure selected from the group consisting of at least about 5 kg/cm², at least about 10 kg/cm², at least about 50 kg/cm², at least about 100 kg/cm², and at least about 500 kg/cm², while permitting flow of ventilating gas through the lumen.

12. An oropharyngeal device according to claim 1 wherein the tube portion is flexible distally and proximally of the bite-resistant zone about an axis transverse to the tube portion and the bite-resistant zone is relatively inflexible about axes transverse to the tube portion.

13. An oropharyngeal device according to claim 1 wherein the tube wall comprises an inner tubular layer and an outer tubular layer contiguous with the inner tubular layer and wherein the resilient wire reinforcement is between the inner and outer tubular layers.

14. An oropharyngeal device according to claim 1 wherein the polymeric material is transparent, the resilient wire reinforcement is entirely embedded within the tube wall and is visible through the tube wall.

15. An oropharyngeal device according to claim 1 wherein the resilient wire reinforcement comprises a material selected from the group consisting of a metal, a metal alloy, a carbon fiber, or combinations thereof.

16. An oropharyngeal device according to claim 1 wherein the flexible resilient polymeric material comprises a medical grade polyvinyl chloride polymer, and the polyvinyl chloride polymer optionally is transparent.

17. An oropharyngeal device according to claim 1 wherein the proximal end comprises an adaptor for connecting with a source of ventilation.

18. An oropharyngeal device according to claim 1 wherein the bite-resistant zone can yield during biting to resist bite-induced injury while maintaining patency of the lumen.

19. An oropharyngeal device according to claim 1 wherein the distal end is configured to communicate ventilating gas into the trachea of the patient and the oropharyngeal device constitutes an endotracheal tube.

20. An oropharyngeal device according to claim 1 wherein the distal end comprises a laryngeal mask to extend over the larynx of the patient and communicate ventilating gas to the pharyngeal airway of the patient and the oropharyngeal device constitutes a laryngeal mask airway.

21. An oropharyngeal device according to claim 1 wherein the resilient wire reinforcement comprises continuous, woven, braided or interlaced wire and the resilient wire reinforcement is configured as a cylinder, the cylinder having a circular, oval, elliptical or “C”-shaped cross-section.

22. An oropharyngeal device according to claim 1 wherein the resilient wire reinforcement comprises one or more resilient wire members having a sinuous, zigzag or other fluctuating configuration the fluctuation being with respect to a circumferential or helical line extending around the tube portion.

23. An oropharyngeal device according to claim 1 wherein the tube portion comprises additional reinforcement distally and proximally of the bite-resistant zone permitting limited flexing of the tube portion about an axis transverse to the tube portion distally and proximally of the bite-resistant zone.

24. An oropharyngeal device according to claim 1 wherein the tube portion comprises a kink-inhibiting structure non-contiguous with the resilient wire reinforcement.

25. An oropharyngeal device according to claim 24 wherein the kink-inhibiting structure comprises a wire, wherein the wire comprises widely-spaced wire turns.

26. An oropharyngeal device for use in ventilating a patient by supplying a ventilating gas to an airway of the patient, the oropharyngeal device comprising:
a proximal end connectable with a source of ventilating gas;
a distal end disposable within the airway of the patient with the proximal end located externally of the patient; and
a tube portion extending between the proximal end and the distal end, the tube portion defining a lumen permitting ventilating gas to flow through the oropharyngeal device from the proximal end to the distal end and comprising:
a tube wall comprising a flexible resilient polymeric material; and
a reinforcement member extending around the lumen in a bite-resistant zone, the bite-resistant zone located intermediately along the tube portion between the proximal end and the distal end and being subject to engagement between the teeth of the patient during use;
wherein the reinforcement member can maintain the patency of the lumen during biting to permit the ventilating gas to flow through the lumen and the tube wall provides cushioning and is yieldable in response to biting pressure.

27. An oropharyngeal device according to claim 26 wherein the reinforcement member is configured as a cylinder, the cylinder having a circular, an oval, an elliptical, or a “C”-shaped cross-section.

28. An oropharyngeal device according to claim 26 wherein the reinforcement member comprises one or more strands, threads, filaments, or fibers of a metallic material, a metallic alloy material, a composite material, a carbon fiber material, or combinations thereof.

29. An oropharyngeal device according to claim 26 wherein the reinforcement member material has a woven, braided or interlaced configuration.

30. An oropharyngeal device according to claim 26 wherein the reinforcement member is embedded in the tube wall.

31. An oropharyngeal device according to claim 26 comprising a distal tube kink-inhibiting structure and a proximal tube kink-exhibiting structure, located distally and proximally adjacent to the reinforcement member, respectively, the distal and proximal tube kink-inhibiting structures being capable of supporting the tube wall to permit flexing of the tube portion about an axis transverse to the tube portion and prevent kinking.

32. A manufacturing process for making an orolaryngeal device, the process comprising:
preforming, preassembling, or predisposing a reinforcement member to a structure suitable for disposition in a bite-resistant zone; and
building a tube portion outside, inside or around the preformed, preassembled or predisposed reinforcement member, by extrusion, by molding, or by deposition to create an orolaryngeal device comprising a lumen, wherein the oropharyngeal device is capable of maintaining the patency of a lumen disposed within the orolaryngeal device during biting by a patient.
